(19) United States
(12) Patent Application Publication
(10) Pub. No.: US 2004/0010264 A1
(43) Pub. Date: Jan. 15, 2004

(54) Cable passer for less invasive surgery

(76) Inventors: Dean Acker, Warsaw, IN (US); Jim Collins, Columbia City, IN (US); Kevin Cook, Warsaw, IN (US); Robert Krebs, Warsaw, IN (US)

Correspondence Address: JACQUE R. WILSON, ESQ., ZIMMER INC., 345 E. MAIN ST., WARSAW, IN, 46580, US

(21) Appl. No.: 10/120,797

(22) Filed: Jul. 15, 2002

(51) Int. Cl. A61B 17/58

(52) U.S. Cl. 606/103

(57) ABSTRACT

A cable passing apparatus useful in passing a cable around a bone. The cable is used to repair broken bone during an orthopaedic surgery. The cable passer comprises a cannulated internal assembly that is extendable at least partially around a bone. A cable suitable for surgical applications can be threaded through the apparatus and removed as desired.

### 1. BACKGROUND OF THE INVENTION

Field of the Invention. The present invention relates generally to orthopaedic surgical instruments and more specifically to surgical instruments for passing cable around bone.

### 2. DESCRIPTION OF RELATED ART

When individuals suffer a relatively severe bone break, orthopaedic surgeons are often called upon to reset the damaged bone. To prevent splintering of the bone such repairs are often accomplished by the surgical insertion of endoprosthetic implants such as stems, nails or other prosthetic devices into bone. During surgery the previously damaged bone may become fragmented or splintered. It is sometimes necessary therefore, for the orthopaedic surgeon to secure fragmented pieces of bone to one another using cable. Regardless of the surgical procedure, the process of using such a cable is much the same. A cable is passed around the bone, drawn tightly, secured, then left in place. The use of such orthopaedic cables to accomplish the task described above is well known.

Surgical instruments useful for passing cable around bone are also well known; however, it has become increasingly important for physicians to perform familiar surgical techniques in a minimally invasive manner. Minimally invasive surgery results in less surgical trauma to the patient. These surgeries require smaller incisions and result in less soft tissue trauma, but accomplish the same patient care goals that transitional, more invasive, surgical techniques achieve. The value of any minimally invasive surgical technique is vast, because damage to a patient's soft tissue is decreased, so too is the time required for the patient to recover from surgery. Thus, a minimally invasive surgery typically results in a faster recovery time for patients. Any minimally invasive surgical technique, however, requires not only a skilled surgeon, but also specially designed surgical tools. Therefore, a need for such surgical instruments is ever present.

Furthermore, passing cable around bone using prior art devices or techniques is cumbersome for the surgeon and not without a traumatic affect to the patient. When using a cable to repair or reinforce bone, for example, the cable passing instrument must be inserted through the skin, either through a new incision or an existing incision, and oriented such that it may pass a cable around a desired bone. The cable is subsequently tightened about the bone and secured. In order to accomplish this task, with minimal trauma to the patient and minimal difficulty for the surgeon, a need exists for a new cable passing surgical instrument.

### 3. SUMMARY OF THE INVENTION

The present invention relates generally to a cable passer for use in standard and in minimally invasive orthopaedic surgeries.

In a preferred embodiment, the cable passer of the present intention comprises a cannulated body, said body comprising a handle portion, a straight shaft portion, and a curved shaft portion; a cannulated push rod slideably disposed within the bore of the cannulated body; a cannulated flexible tube connected to one end of the push rod and slideable within the body; and a curved cannulated needle connected to one end of the flexible tube. The bore of each interior piece is concentric, such that a cable may be inserted therethrough, from either end of the apparatus to accommodate different cables or different surgical techniques. Thereafter, one can remove the cable passer while leaving the cable in place, then secure the cable to the bone, as desired. In addition, the cable passer of the present invention comprises a modular design suitable for disassembly, thereby making the device suitable for easier cleaning.

### 4. BRIEF DESCRIPTION OF THE DRAWINGS

FIG. 1 is a perspective view of a cable passer according to the present invention.

FIG. 2 is a longitudinal cross sectional view of the cable passer shown in FIG. 1.

FIG. 2a is a longitudinal cross sectional view of the cable passer shown in FIG. 1, with the needle extended.

FIG. 3 is a cross sectional view of the push rod component of the present invention.

FIG. 4 is a cross sectional view of the connector of the present invention.

FIG. 5 is a cross sectional view of the flexible tube component of the present invention.

FIG. 6 is a cross sectional view of a curved needle according to the present invention.

FIG. 7 is an elevational view of a cable passer according to the present invention with the cable passer shown adjacent to a bone.

FIG. 8 is an elevational view of a cable passer according to the present invention with the cable passer needle extended around a bone.

FIG. 9 is a cross sectional view of the handle of the present cable passer according to a preferred embodiment.

FIG. 10 is a side elevational view of the ball spring shown in FIG. 9

FIG. 11 is a side elevational view of the push rod of the present cable passer according to a preferred embodiment.

### 5. DETAILED DESCRIPTION OF THE INVENTION

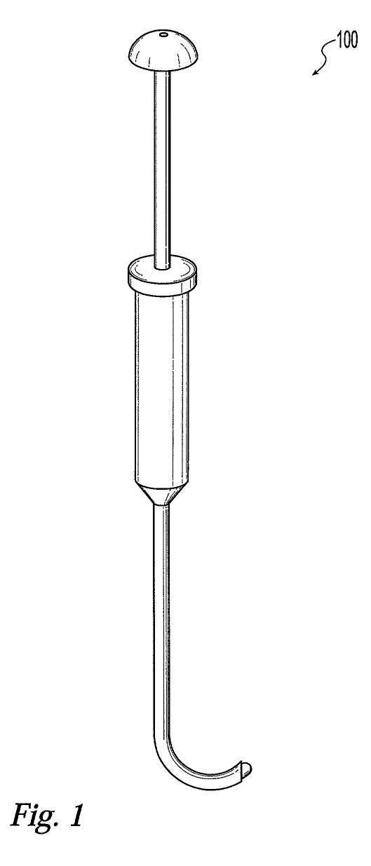

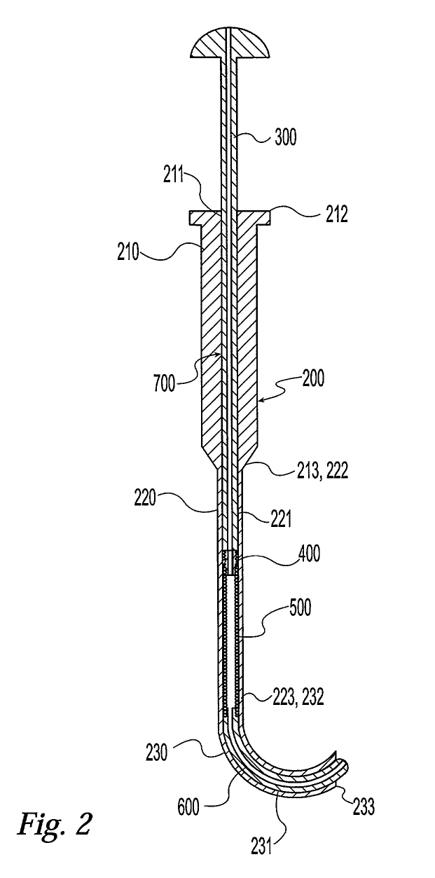

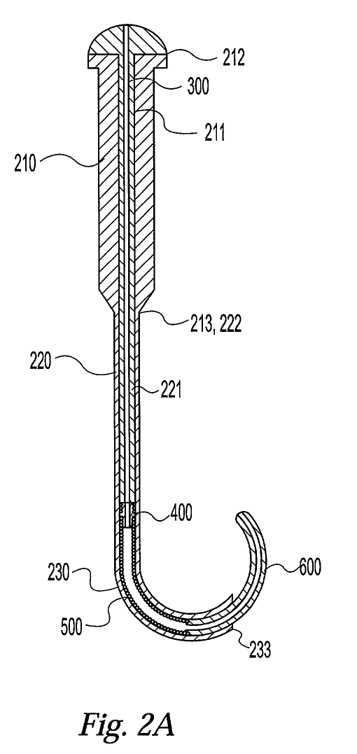

FIG. 1 shows a perspective view of an apparatus according to the present invention, cable passer 100. As shown in FIGS. 2 and 2a, cable passer 100 comprises assembly 700 slideably disposed within body 200. Assembly 700 comprises push rod 300, connector 400, flexible tube 500, and arcuated cannulated needle 600. The modular design of cable passer 100 allows for the instrument to be more easily cleaned. Alternatively, the cable passer of the present could be disposable rather than reusable.

Referring still to FIG. 2 or FIG. 2a, body 200 is preferably formed of stainless steel, but may be formed of any bio-compatible material. In the preferred embodiment of the present invention, body 200 comprises handle 210, straight shaft 220, and arcuated tube 230. Handle 210 is generally cylindrically shaped. Handle 210 comprises bore 211 along its longitudinal axis, and opposing ends 212 and 213. Handle 210 also preferably comprises a textured exterior surface to facilitate an operator's handling of the present invention. The walls of bore 211 are substantially smooth, and the cross sectional area of bore 211 is large enough to slidably accommodate assembly 700, which assembly 700 is described in greater detail herein below.

In FIGS. 2 and 2a, there is also shown shaft 220. Shaft 220 has a generally cylindrical shape and opposing ends 222 and 223. Shaft 220 comprises bore 221 disposed along the longitudinal axis of shaft 220 such that bore 221 is in communication with each of the opposing ends 222 and 223 of shaft 220. Opposing end 222 of shaft 220 is integrally attached to end 213 of handle 210, such that bore 221 is concentric with bore 211. Preferably, both handle 210 and shaft 220 are machined from a single metal piece. Bore 221 comprises substantially smooth walls, and the cross sectional area of bore 221 is large enough to slidably accommodate assembly 700. Bore 221 is preferably equal in diameter to that of bore 211. Integrally attached to end 213 of shaft 220 is arcuated tube 230.

Handle 210, shaft 220, and tube 230 may be machined from a single metal piece. Referring still to FIG. 2, acruated tube 230, having a generally cylindrical shape, comprises bore 231 and ends 232 and 233. End 232 is preferably integrally attached to end 223 of shaft 220. Bore 231 is disposed along the longitudinal central arc of tube 230, such that bore 231 is in communication with ends 232 and 233 of tube 230 and with bore 221 of shaft 220. Bore 231 comprises substantially smooth walls and a cross sectional area preferably of equal size to that of bore 221 in shaft 220. Bore 231 is also large enough to slidably accommodate assembly 700. End 233 of tube 230 remains free.

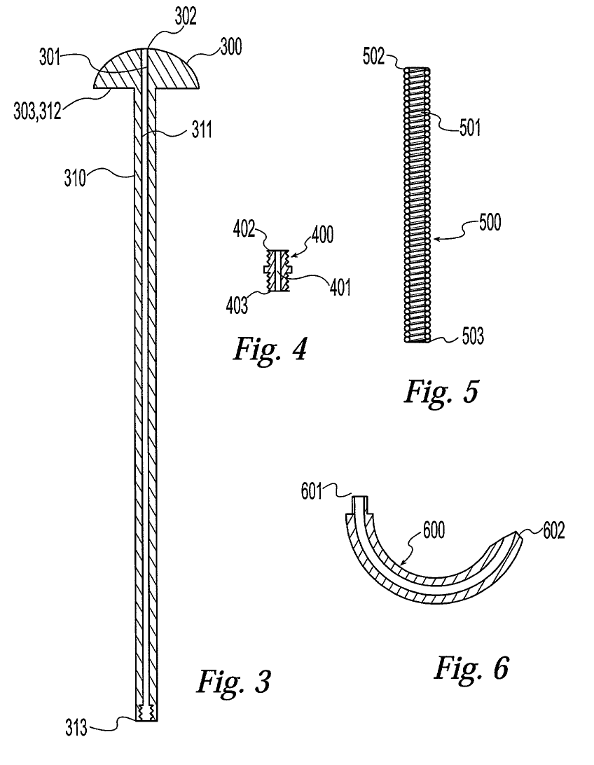

Referring again to FIGS. 2 and 2a, assembly 700 is slidably disposed within body 200 of the present invention. As shown in FIGS. 4-7, assembly 700 comprises cannulated push rod top 300, cannulated push rod 310, cannulated connector 400, cannulated flexible tube 500, and cannulated curved needle 600. Referring now to FIG. 3 there is shown cannulated push rod top 300. Top 300 preferably comprises a cylindrical disc or hemisphere having a desired height and diameter, wherein the height is less than the radius. Preferably the radius of Top 300 is greater than the radius of handle 210. Top 300 further comprises bore 301 disposed along the longitudinal axis of top 300 and opposing ends 302 and 303. Ends 302 and 303 are in communication with bore 301.

Referring again to FIG. 3, fixedly attached to top 300 is cannulated push rod 310 having bore 311 and opposing ends 312 and 313. Push rod 310 preferably has a cylindrical shape. Push rod 310 is preferably composed of stainless steel or another bio-compatible material. Bore 311 is disposed within push rod 310 such that bore 311 extends along the longitudinal axis of push rod 310 and is in communication with opposing ends 312 and 313 of push rod 310. End 312 of push rod 310 is fixedly attached to, and extends perpendicularly from, end 303 of top 300 such that bore 301 and bore 311 are concentric. Opposing end 313 of push rod 310 remains free.

Referring now to FIG. 4, there is shown cannulated connector 400. Cannulated connector 400 is generally cylindrically shaped and has a threaded exterior. Cannulated connector 400 comprises bore 401 and opposing ends 402 and 403. Bore 401 is disposed along the longitudinal axis of connector 400 such that bore 401 is in communication with opposing ends 402 and 403. As shown in FIGS. 2 and 2a, end 402 of connector 400 is threadibly connected to end 313 of elongated member 310 such that bore 401 is concentric with bore 311 of push rod 310.

Referring now to FIG. 5, flexible tube 500 is preferably cylindrically shaped and composed of stainless steel or another biocompatible material. Flexible tube 500 may comprise a tubular spring; however, the preferred embodiment comprises a metal tube having laser-cut spirals. These spirals provide flexibility for tube 500. Tube 500 further comprises bore 501 and opposing ends 502 and 503. Bore 501 is disposed along the longitudinal axis of tube 500 such that it is in communication with ends 502 and 503 thereof. End 502 is fixedly connected to end 403 of connector 400 such that bore 501 is concentric with bore 401 of connector 400, as shown in FIGS. 2 and 2a.

As shown in FIG. 6, curved needle 600 is an arcuated tube having a single radius. Needle 600 is composed preferably of stainless steel. Needle 600 further comprises bore 601 and opposing ends 602 and 603. Bore 601 extends through the center of needle 600 along an arc of uniform radius such that bore 601 is in communication with ends 602 and 603. As shown in FIGS. 2 and 2a, end 602 of needle 600 is fixably attached to end 503 of flexible tube 500 such that bore 601 is in communication with bore 501 of tube 500. End 603 of needle 600 comprises a pointed tip suitable for insertion into or through soft human tissue.

Referring again to FIG. 2a, there is shown the assembly 700, comprising of push rod top 300, push rod 310, connector 400, flexible tube 500 and needle 600 as described herein. Assembly 700 is slideably disposed within bores 215, 225 and 231 of body 200 In addition, bores 301, 401, 501 and 601 of assembly 700 are of sufficient diameter to accommodate a surgical cable being inserted therethrough.

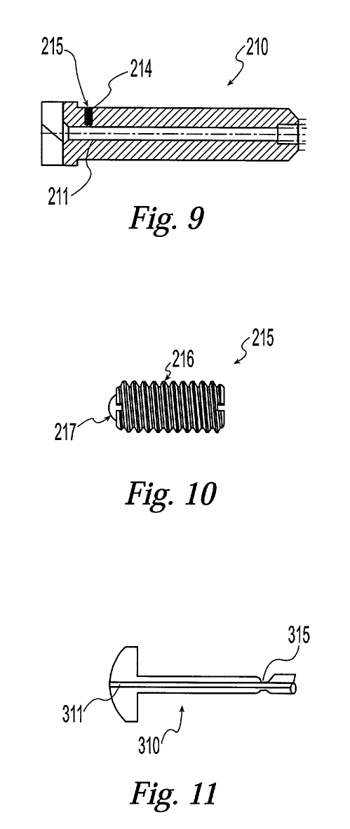

Referring now to FIG. 9, in a preferred embodiment of the present invention, handle 210 further comprises bore 214. Bore 214 is disposed through the wall of handle 210 such that bore 214 is in communication with bore 211 of handle 210 and with the outside of handle 210. Ball spring 215 is slideably disposed within bore 214. Ball spring 215 comprises spring 216 and plunger 217 as shown in FIG. 11.

In this preferred embodiment, as shown in FIG. 11, push rod 310 comprises a notch 315 disposed therearound at a desired location. The notch 315 is suitable for the removable insertion of plunger 217 as the push rod 310 is moved through bore 211 of handle 210.

A user of the present cable passer will be able to feel ball spring 215 insert itself into notch 315. Thus, the cable passer will provide its user tactile-feedback of how far the user has pulled the push rod 310 out of handle 210.

It will be appreciated by those skilled in the art that the foregoing is a description of a preferred embodiment of the present invention and that variations is design and construction may be made to the preferred embodiment without departing from the spirit and scope of the present invention as defined by the appended claims.

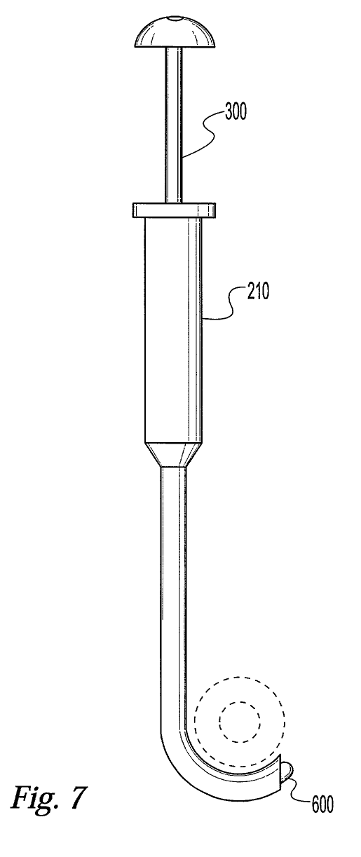

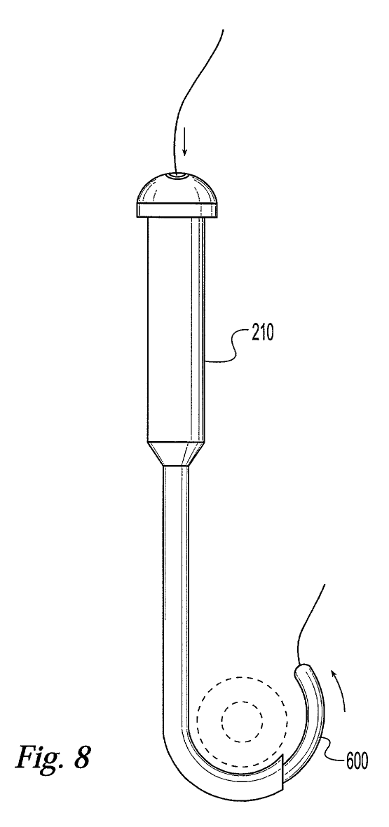

Referring now to FIGS. 7 and 8, to use the device described herein, the surgeon retracts push rod top 300 such that curved needle 600 of assembly 700 is contained within tube 230 of body 200. End 233 of tube 230 is then inserted into a patient as desired by the surgeon. Top 300 is then depressed such that needle 600 protrudes from the tube 230 of body 200. Thereafter, the surgeon may insert a surgical cable into bore 301 of top 300 or into end 603 of needle 600. The surgical cable is continually threaded into the chosen end of the apparatus until a portion of the cable protrudes from the unchosen end of the apparatus. Thereafter, the cable is held in place and the apparatus is removed from the patient's body, while leaving the cable in place. At this point, other means well known to those skilled in the art, are used to secure the cable around the desired bone in order to provide the support needed to the damaged bone or surgical site.

1. An apparatus for passing a cable around a bone, the apparatus comprising:
(a) A cannulated body of desired shape, the body comprising a straight portion, an arcuated portion, a first end, a second end, and a bore disposed there between such that the first end is in communication with the second end;
(b) An assembly slideably disposed within the body, the assembly comprising:
i. a top having a shape, a first end, a second top end, and a bore disposed between the first top end and the second top end such that the first top end is in communication with the second top end, the second top end fixedly attached to the flexible tube such that the tube bore is in communication with the top bore, the top disposed outside of the body.
ii. a cannulated flexible tube having a first tube end, a second tube end, and a tube bore disposed there between such that the first tube end is in communication with the second tube end;
iii. an arcuated cannulated needle disposed within the arcuated portion of the body, the needle having a first needle end, a second needle end and a needle bore disposed there between such that the first needle end is in communication with the second needle end, the first needle end fixedly attached to the second tube end such that the needle bore and tube bore are in communication, the second tube end extendable from the second body end.

2. The apparatus of claim 1, wherein the straight portion is a handle comprising a textured surface.

3. The apparatus of claim 1, wherein the flexible tube is a metal tube having laser-cut spirals.

4. The apparatus of claim 1, wherein the flexible tube comprises a metal spring.

5. The apparatus of claim 1, wherein the second end of the needle further comprises a pointed tip.

6. The apparatus of claim 1, wherein the body is comprises of stainless steel.

7. The apparatus of claim 1, wherein the assembly is comprises of stainless steel.

8. The apparatus of claim 1, wherein the straight body portion and the acruated body portion are machined from a single metal piece such that the straight portion and the arcuated portion are integrally connected.

9. The apparatus of claim 1, wherein the straight body portion and the arcuated body portion are welded together.

10. The apparatus of claim 1, wherein the body comprises a generally cylindrical shape.

11. The apparatus of claim 1, wherein the assembly comprises a generally cylindrical shape.

12. The apparatus of claim 1, wherein the top comprises a cylindrical shape having a height less than its diameter.

13. The apparatus of claim 1, wherein the top comprises stainless steel.

14. The apparatus of claim 1, wherein the assembly further comprises a cannulated push rod, the push rod slideably disposed within the body, the push rod comprising the same shape as the body, a first push rod end, a second push rod end and a push rod bore disposed there between such that the first push rod end is in communication with the top the second push rod end, the first push rod end extendable out of the body, the first push rod end fixedly attached to the second top end such that the push rod bore is concentric with and in communication with the top bore, the second push rod end fixedly attached to the first flexible tube end such that the tube bore is concentric and in communication with the push rod bore.

15. The apparatus of claim 14, wherein the second push rod end is releasably attached to the first end of the flexible tube.

16. The apparatus of claim 14, wherein the second push rod end is threadably attached to the first end of the flexible tube.

17. The apparatus of claim 14, wherein the push rod comprises stainless steel.

18. The apparatus of claim 14, wherein the assembly further comprises a cannulated connector, the connector slideably disposed within the bore of the body, the connector having a first connector end, a second connector end, and a connector bore disposed there between such that the first connector end is in communication with the second connector end, the first connector end attached to the second push rod end such that the connector bore is concentric with and in communication with the push rod bore, the second connector end fixedly attached to the first tube end such that the connector bore is concentric with and in communication with the tube bore.

19. The apparatus of claim 18, wherein the first connector end is releasably attached to the second push rod end.

20. The apparatus of claim 19, wherein the first connector end is threadably attached to the second push rod end.
